(19) United States
(12) Patent Application Publication
(10) Pub. No.: US 2019/0380682 A1
(43) Pub. Date: Dec. 19, 2019

(54) Pressure Wave Shaping Metasurface

(71) Applicants: Paolo Menegoli, San Jose, CA (US); Fabio Alessio Marino, San Marcos, CA (US)

(72) Inventors: Paolo Menegoli, San Jose, CA (US); Fabio Alessio Marino, San Marcos, CA (US)

(21) Appl. No.: 16/433,395

(22) Filed: Jun. 6, 2019

Related U.S. Application Data
(60) Provisional application No. 62/685,463, filed on Jun. 15, 2018.

(51) Int. Cl. A61B 8/00; G01S 15/89; A61B 8/14; A61N 7/02

(52) U.S. Cl. CPC A61B 8/4483; G01S 15/89; A61N 2007/0056; A61N 7/02; A61B 8/145

(57) ABSTRACT

The present invention describes a method to shape and control acoustic and pressure waves through to the use of metasurfaces applied to the waves. In particular the present invention describes a method to make the use of metasurfaces, for long wavelength waves, like the sound and haptic waves, convenient and practical by up-converting to higher frequency the original signal. Ultrasonic waves reduce the size of the metastructures within the metasurfaces to become small enough to open new frontier in the control of acoustic and pressure waves. The metasurfaces can be made tunable to widen even more the possible applications for metasurface technologies.

### FIELD OF INVENTION

The present disclosure relates to the field of metasurfaces. The subject application also relates to the field of pressure wave shaping and processing. The subject application also relates to the field of audio and acoustic applications.

### BACKGROUND

Metasurfaces and metamaterials are consisting of periodic nanostructures built on a medium to shape the response of a wave. These nanostructures are built with dimensions smaller than the wavelength, to modify the path or the wave-front of the wave, similarly to changing the refractive index of a material. For example the angle of an emitting wave could be varied such as to be more directly directed to a desired target. If the metasurfaces are made tunable, an adaptive system can change the physical properties of the incident wave (phase, amplitude and polarization) to obtain a desired path for the wave. For example the wave can be focused at different distance by modulating the metasurfaces.

Generally, and more commonly, metasurfaces are utilized and analyzed for electromagnetic waves, however, in theory, they can also be applied to waves of different nature. For example to magnetic waves or acoustic waves. In particular the subject application focuses on the acoustic and, more in general, to pressure waves.

It has been demonstrated that acoustics follows physics laws that are analogous to the ones of the electromagnetic field, where mass density and bulk modulus are analogous to the magnetic permeability and to the electric permittivity of the EM field.

Metasurfaces engineer the response to a wave and ideally can alter the impedance of the signal at a medium interface. Therefore almost ideal absorbers or reflectors can be implemented. In fact one could envision an acoustic absorber made of a metasurface to be applied to a surface (for example a wall of a building) to suppress or absorb certain undesired acoustic noise.

Similarly a metasurface can be applied to a wall to reflect audio waves at certain frequencies, for instance to improve hearing/acoustics where a television set is located, without requiring multiple speakers. Moreover a metasurface can be made of a sheet material where an absorbing metasurface is applied to one side and a reflecting one is applied to the other one.

Generally metasurfaces can be made one order of magnitude smaller than the wavelength, even though it has been recently demonstrated that metasurfaces for acoustic purposes, with dimensions two order of magnitude smaller than the wavelength, are also effective. The audible frequencies span is from about 20-50 Hz to about 15 KHz-20 KHz. If one restricts this range between 100 Hz and 10 KHz (where generally human voice stands) the wavelength is between 34 mm and 3.4 m, therefore if the size of the metastructures can be about 10 times smaller than the wavelength, they would range in size between 3.4 mm and 340 mm.

Therefore there is a need to be able to control a mechanical wave (acoustic or pressure wave) using metasurfaces or similar periodic nanostructures to focus a beam, or to reflect a large portion of the wave or to control the phase of a wave to create directional transmission as for the case of beam forming, however generally the size of these structure would be prohibitively large to make the technology practical in most acoustic or pressure based applications.

### BRIEF SUMMARY OF THE INVENTION

The present invention describes several methods of applying metasurfaces to mechanical, acoustic and pressure based waves so as to engineer their front, direct the waves, control their phase, absorb them or reflect them depending on the specific requirements of the respective applications. The present invention further describes a method to transfer acoustic and pressure based waves to higher frequency so as to make the signal wavelength much shorter and therefore the size of the applied metasurface significantly smaller.

This subject application explores various possible applications and utilizations of metasurfaces implemented in acoustic and pressure wave fields.

### DETAILED DESCRIPTION OF THE INVENTION

The present invention will now be described in detail with reference to certain embodiments thereof described. In the following description, numerous specific details are set forth in order to provide a thorough understanding of the present invention. It will be apparent, however, to one skilled in the art, that the present invention may be practiced without some or all of these specific details. In other instances, well known details have not been described in detail in order not to unnecessarily obscure the present invention.

In the field of electromagnetic waves (radio frequencies for cellular phones or radio and TV broadcasting) frequency modulation techniques have been used for a long time. That offers many advantages like increased signal bandwidth and noise free reception. Acoustics parametric loudspeakers have been invented many years ago, based on up-conversion in frequency of the audio signal by means of frequency modulation of the audio signal onto an higher frequency carrier. Ultrasounds tends to disperse less due to their higher frequency, for the same emitter size, therefore they carry the signal farther. Any non-linearity, that normally occurs in the sound wave path, for example any obstacle, demodulates the signal into its harmonics, and if the difference between the two main transmitted frequencies is the audio signal frequency, that signal is transferred into the human audible spectrum and is therefore audible by a person.

One embodiment of this invention is a loudspeaker to reproduce sound combined with a metasurface to direct, or focus, the sound in a specific direction/spot. The higher frequency carrier signal makes the use of one or more metasurfaces practical and efficient, since their size becomes very small. In addition in this case the directivity of the signal is increased significantly for effect of the metasurface (which could act as an acoustic lens) and for effect of the ultrasonic transmission of the mechanical sound wave, which by itself allows an increase of the distance reached by the wave by more than 20 times with respect to conventional sound reproduction.

In another embodiment of the present invention the modulated ultrasound signal is at much higher frequency, therefore requiring an even smaller metasurface structure size (since that it is related to the wavelength of the signal being processed). If for example the ultrasound is chosen to be at 1 MHz, its wavelength is about 0.35 mm and therefore the metastructure could be made significantly smaller than what it would be without the ultrasound frequency modulation of the audio signal, making the technology appealing for small speaker applications. Therefore the combination of ultrasound modulation and metasurfaces is employed to implement very small, cost effective and quite directional transducers.

In another embodiment of the present invention a metasurface is used to increase directionality to a smart phone speaker, for example to add privacy to a speakerphone of a smart phone, where only the user in front of it can hear its sound. This implementation again combines focusing metasurfaces with the frequency up-conversion of the audio signal by modulating the audio signal with an ultrasonic carrier of desired frequency. In addition to obtain higher directivity, the speaker dimensions are reduced. One disadvantage of the conventional parametric speaker approach is that the low frequency sounds get generally cut off. However, in the case of the smart phone applications, or small electronic devices in general, and in particular for the case of the speaker-phone the bass sounds are not well reproduced anyway.

Generally when these techniques are applied to up-conversion of the audio signal a significant amount of pre-processing and possible pre-distortion of the signal is required. Nowadays any smart phone device has all the digital hardware and software capabilities to apply such pre-processing within the device itself.

One technical challenge of the metasurfaces in general is their inherent low bandwidth, and in fact it is mostly utilized as a monochromatic device. However, although for optical signals in the visible range the human eye is capable of viewing several frequencies at the same time, and in fact any color in the visible spectrum can be synthetically reproduced by the combination of three or more colors, in the case of the human ear, the reproduced audio signal superimposes the various harmonics and appears as one signal (which is the signal that generally is managed by a loudspeaker) that contains all the harmonics (which get separated by our ear and brain). It should be mentioned that the up-conversion of the audio signal to the frequency carrier also makes any variation of the audio signal smaller in frequency with respect to the carrier frequency, whether the signal is amplitude modulated or frequency modulated.

However to overcome this bandwidth limitation, and in another embodiment of the present invention, the metasurface may be made tunable, and if the tuning technique is fast enough (at least as fast as the audio envelope) that the metastructures can be varied in shape and size in synchronism with the modulated audio signal (which is added to the ultrasound frequency), one or more metasurfaces could be made almost monochromatic for each audio frequency. Assuming that any audio signal could be generated as sum of two (or more) signals at a different and given frequency, then two (or more) metasurfaces in series could operate to be effective for the corresponding two (or more) wavelengths at any time and the tuning of the metasurfaces could vary the size and/or shapes of the metastructures so as to be operating effectively at each instantaneous frequency. By varying the shape and size of the metasurfaces, the wavelength, at which each metasurface is effective, varies, and by superimposing the effects of two (or more) of such metasurfaces any audio signal could be represented and processed by the real time modulating metasurfaces.

In another embodiment of the subject invention this technique can be combined with the use of beam forming using multiple audio transducers or transmitters to provide constructive interference to the ultrasound signal, thus augmenting further the directionality of the speaker. If the metasurface or the phase of an array of speakers can be varied, then the directionality of the sound beam can be modulated as desired, for instance to track the movement of a listening target. For example if a system can determine where the sound is coming from, it can direct the responsive sound to the same direction of the incoming sound. This could be implemented in home systems like the current Echo or Alexa from Amazon, smart speakers with voice-controlled intelligent assistant service devices. In addition the system can direct the ultrasonic beam to a specific area so as to make it bounce out of a surface or object to change in real time the direction of the reflected source, as to make it sound like the signal source is moving.

The metastructures can be tuned by utilizing several methods, for instance by using piezoelectric actuators, or MEMS or any other actuating method. In one embodiment of the subject invention a metasurface is digitally tunable. The metasurface could be tuned between two different states (typically two extremes with respect to the desired variation of the tunability) and a PWM system can toggle at high enough frequency (in most applications it does not have to be a high frequency) between these two states to effectively obtain any value between the two states. If the tuning of the metasurface is to track an audio signal, the digital tuning has to be fast enough to prevent aliasing of the signal, for example at least 10 times faster than the audio signal, however generally the tuning of the metasurface can be employed to change directionality of a speaker, or of a beam, or to focus the beam at a varying distance from the source. These applications generally do not require high tuning bandwidth.

In alternative to the described audio applications, this system could be used to direct sound to a desired listener in an vehicle (car, bus, train, airplane and others) or in any other environment (shopping mall, in a street, or in a working environment). Or it could be used to blast a high power acoustic wave directed to an intruder or a burglar without disturbing the neighbor or any person within a given distance. It could also be used by law enforcement agencies to diffuse violent or dangerous situations without resorting to the use of firearms or other harmful weapons.

A similar concept can be used for haptics, and more in general for pressure wave based feedback systems. These systems are generally monochromatic (single frequency) therefore they are simpler to implement with metasurfaces with respect to audio applications that require a certain bandwidth of the signal. However, also in this case, the signal frequency for these applications is generally quite low, because the human tactile sensory bandwidth is generally considered to be in the range of 20 Hz to 200 Hz, therefore, again, it is advantageous to modulate these signals with higher frequency carriers (for example ultrasounds at hundreds of KHz or even at several MHz) to obtain smaller metastructures. The metasurfaces can focus the haptic signal and therefore apply more force to the sensor location effectively making the system more efficient, but it can also reduce the number of haptic transducers or transmitters if the metasurface is made tunable.

It is therefore another embodiment of the present invention to have a haptic system, for example for virtual reality (gaming applications), using metasurfaces. It is also another embodiment of the present invention to have a haptic system for adaptive Braille reading systems (a system that stimulates the fingers of a blind individual) to replicate the Braille characters of a virtual keyboard in air, using metasurfaces.

It is another embodiment of the present invention to have a communication system that reproduces, transmits and receives the touch sensation of an object, or animated being, by means of touch sensors and haptic transducers using higher frequency modulation and adaptive metasurfaces. This could allow the virtual sensation of remotely touching an object or a person via computer or via portable communication device.

It is another embodiment of the present invention to have an acoustic levitation system that uses metasurfaces to adaptively control the levitation of an object when the levitated object is in motion.

Acoustic levitation is based on generating a standing pressure wave by having the distance between a source and a reflector exactly equal to a multiple of the wavelength, thus having periodic areas of maximum pressure and minimum pressure. At the nodes of maximum pressure the object can balance the pressure with its own weight and therefore levitate. However, as soon as the distance between the source and the reflector is varied, for any reason, the standing wave is no longer a standing wave and the object is no longer sustained by the acoustic pressure. When an object is intended to be moved from one location to another, by means of acoustic levitation systems (for example in systems where contamination or any human handling is not allowed), it is important to move along the standing wave in order to generate the desired object movement. Although there are other methods, a tunable metasurface, positioned between the ultrasound source and the reflector, can change the physical parameters of the pressure waves to generate the object's movement without dropping it. These systems are used in the pharmaceutical and electronics industry for container-less processing.

Another area of interest is the field of energy scavenging and wireless power transfer in general. By using ultrasound emitters and ultrasound transducers or receivers that convert the acoustic/pressure waves into electric energy, it is possible to transfer power from one source to a remote device. For example this method could be used for powering up or charging implanted devices located within the human body. Ultrasounds are known to be safe, if used within a specified power level, therefore it is possible to provide energy to a small implanted device by means of ultrasounds. It is therefore another embodiment of the present invention a wireless power transfer system where a metasurface allows the focusing of an ultrasound beam to a specific area (typically where the ultrasound transducer/receiver is located, for example the implanted device) with the intent to transfer power efficiently to the receiver.

By focusing the ultrasound beam directly onto the receiver the efficiency of the system is improved. If the receiver is moving with respect to the acoustic transmitter (for instance sensors within smart ingested pills that move within the digestive system or within the vascular system) the system, according to the disclosed invention, can comprise the ability to track the object and re-focus or re-direct the beam by means of tunable metasurface.

Another embodiment of the present invention is in the medical field. Nowadays diagnostic devices based on ultrasounds are very common, because they are relatively cheap, immediate, painless and non invasive. The resolution of echo-graph systems is dependent on the power emitted by the ultrasound source and the ability to discern accurately the reflected waves. The resolution of these systems has improved throughout the years, but it is still quite limited due to the intrinsic limitations of the ultrasounds. Higher frequencies increase the directivity of the beam, but their penetration is reduced; lower frequencies, on the contrary, allow deeper penetration, but reduce the directivity and therefore increase the scattered reflected waves.

Metasurfaces allow the creation of ultrasonic lenses thus focusing the beam onto the target tissue, therefore reducing the beam diffraction and also its undesired reflections coming from the objects or tissues surrounding the organ under examination. The inability to focus the beam onto the target is one of the main limitations of ultrasound imaging systems to their resolution, since its spatial resolution is generally better than MRI and/or CT.

Furthermore, if the metasurface is made of two layers, each one capable of processing the incoming wave differently in the main outgoing direction (towards the organ under examination) and in the reflected one (back towards the ultrasound detector), the reflected wave can be processed to minimize the undesired scattered ultrasounds, thus focusing the ultrasound detector on the desired echo signal so as to obtain a better definition of the tissue under analysis.

Another embodiment of the present invention is related to the use of metasurfaces to improve the High Intensity Focused Ultrasound (HIFU). HIFU is a relatively new technique to treat some types of tumors by destroying the tumor cells with a localized focused ultrasound beam. The beam heats up the cells (to about 65 to 85 degrees C.) to the point of creating necrosis of the tumor's cells. The beam is focused onto the target by an acoustic lens. The lens is implemented by phase arrays or geometrical lens (spherical curved transducers). However these methods of focusing the beam are quite large in dimensions. A metasurface can significantly reduce the device size, potentially being able to combine this method also with small body incisions. One example where the device size is important is the treatment of the prostate cancer, where the HIFU probe is inserted in the rectum of the patient. Making the device more compact will enable new frontiers to its use.

The typical ultrasound frequency for these applications is between 250 KHz and 2 MHz. At 1 MHz the wavelength is in the order of 350 um (microns) therefore the typical metasurface structure size is in the order of 35 um. This would allow the use of only one metasurface (which could be very small at these frequencies) to focus the beam instead of employing an array of multiple emitting transducers.

Another embodiment of the present invention is related to hearing aids. Hearing aids are generally composed of a microphone, an amplifier and a speaker. Also in this case the possibility to emit ultrasounds modulated at the audio frequencies could allow the use of a metasurface to focus the beam and to make the speaker/transducer very small. Size and efficiency are very important for hearing aid devices. By using ultrasounds, a processing action beyond the amplification is required (for instance pre-distortion modulation may be necessary), however with an acoustic lens the amplification could be minimum and a modulator and a metasurface can be integrated onto a chip in a very compact fashion. The metasurface removes the need to use multiple ultrasound transducers.

Another embodiment of the present invention is related to the use of ultrasounds to heat up food. Ultrasounds are already used in the food industry to process, preserve and extract elements of food in the most efficient and fast way. However generally ultrasounds are not used to cook or bake food. Ultrasound ovens could be an alternative to microwave ovens with the advantage that no electromagnetic waves are generated (safer device) and any object could be safely placed in the oven (even metallic ones). Intense ultrasounds have the ability to heat up the molecules of the material in different way with respect to microwaves. For instance bread cannot be effectively baked with microwaves, while it can be efficiently baked with ultrasounds.

Also in this case focusing the ultrasonic beam onto the food is important to have high efficiency. The use of adaptive or tunable metasurfaces for ultrasonic ovens is beneficial for instance to control and regulate the internal temperature of the cooking food or to control the ultrasonic beam. It is therefore another embodiment of the present invention to use metasurfaces to focus ultrasound beams in a heating chamber (oven) to heat up, cook or bake food in general.

Another embodiment of the present invention is related to the field of sonar to detect targets in a fluid. Sonar operates at frequencies that range from the infra-sounds (a few Hz) to the ultrasounds (a few tens of KHz). At low frequencies using a metasurface would imply large metastructures, nevertheless for sonar applications size may not necessarily be a problem. Again a metasurface for these applications can allow the focusing of the beam, once the target has been identified (a sort of acoustic zoom in), so as to achieve better imaging resolution, and also it can process the reflected beam so as to better isolate the desired echo signal from the undesired scattered noise. The metasurface would have to behave differently in the two opposite sound directions.

Although the present invention has been described above with particularity, this was merely to teach one of ordinary skill in the art how to make and use the invention. Many additional modifications will fall within the scope of the invention. Thus, the scope of the invention is defined by the claims which immediately follow.

As is clear to those skilled in the art, this basic system can be implemented in many specific ways, and the above descriptions are not meant to designate a specific implementation.

What is claimed is:

1. A method for controlling acoustic waves comprising:
up-converting to higher ultrasonic frequency a signal by means of modulation of said signal with an ultrasonic carrier;
transmitting said modulated signal with at least one transducer or transmitter over a physical medium so as to create high frequency acoustic waves;
processing said acoustic waves with at least one metasurface;
controlling amplitude or phase of at least a portion of said acoustic waves with said at least one metasurface;
wherein said at least one metasurface is comprising a plurality of metastructures.

2. The method of claim 1 wherein at least one of said acoustic waves is used in a loudspeaker to focus or direct said signal onto a desired target,
and wherein said signal is an audio signal.

3. The method of claim 1 wherein said acoustic waves are used in a haptic transducer to focus or direct said signal onto a desired target,
and wherein said signal is a haptic signal.

4. The method of claim 1 wherein said at least one metasurface is a tunable metasurface, and wherein a tuning mechanism of said tunable metasurface allows control of phase and amplitude of said acoustic waves.

5. The method of claim 1 wherein said at least one metasurface is a tunable metasurface, and wherein a tuning mechanism of said tunable metasurface allows control of said metastructures of said tunable metasurface in order to vary the wavelength of said acoustic waves at which said tunable metasurface is most effective.

6. The method of claim 1 wherein said acoustic waves are used in a loudspeaker of a portable communication device to focus or direct said signal onto a desired target,
wherein said signal is an audio signal,
and wherein said at least one metasurface and said ultrasonic carrier control the directionality of said audio signal.

7. The method of claim 1 wherein said at least one metasurface allows control of the phase of said acoustic waves,
and wherein said control of said phase implements a beam forming system when a plurality of acoustic waves are emitted by one or more of said transducers.

8. The method of claim 1 wherein said acoustic waves are used to focus or direct said signal onto a moving desired target,
wherein said at least one metasurface is a tunable metasurface,
and wherein a tunable mechanism of said tunable metasurface is used to track said moving desired target.

9. The method of claim 1 wherein said acoustic waves are used to focus or direct said signal onto a moving target,
wherein said at least one metasurface is a tunable metasurface,
wherein a tunable mechanism of said tunable metasurface is used to track said moving desired target,
wherein said moving desired target is a haptic transducer,
and wherein said method is used to communicate remotely the sensation of touch of any object or animated being.

10. The method of claim 1 wherein said at least one metasurface is a tunable metasurface,
wherein said method is used for acoustic levitation systems,
and wherein a tuning mechanism of said tunable metasurface allows control of levitation of an object.

11. The method of claim 1 wherein said method is used for wireless transfer power systems,
wherein energy of said acoustic waves is transferred to a remote receiver,
and wherein said acoustic waves are focused and directed towards said receiver by means of control of said at least one metasurface.

12. The method of claim 1 wherein said method is used for wireless transfer power systems,
wherein said at least one metasurface is a tunable metasurface,
wherein energy of said acoustic waves is transferred to a remote moving receiver,
and wherein said acoustic waves are adaptively focused and directed towards said moving receiver by means of control of said tunable metasurface.

13. The method of claim 1 wherein said method is used for hearing aids systems to focus or direct said signal to a desired direction,
and wherein said signal is an audio signal.

14. A method for controlling ultrasonic waves comprising:
transmitting said ultrasonic waves with one or more transducers over a medium;
processing said ultrasonic waves with at least one metasurface;
controlling amplitude or phase of said ultrasonic waves with said at least one metasurface;
wherein at least one metasurface is comprising a plurality of metastructures.

15. The method of claim 14 wherein said method is used for ultrasound medical imaging systems,
wherein said ultrasonic waves are focused and directed towards a desired target by means of control of said at least one metasurface,
and whereby said method increases the resolution of a generated ultrasound image.

16. The method of claim 14 wherein said method is used for ultrasound medical imaging systems,
wherein said ultrasonic waves are focused and directed towards a desired target by means of control of a first metasurface,
wherein reflected ultrasonic waves are detected by sensors of said ultrasound medical imaging system,
wherein said reflected ultrasonic waves are controlled by means of a second metasurface,
and whereby said method increases the resolution of the ultrasound images generated.

17. The method of claim 14 wherein said method is used for High Intensity Focused Ultrasound systems,
wherein said ultrasonic waves are focused and directed towards a desired target by means of control of said at least one metasurface.

18. The method of claim 14 wherein said method is used for heating up food or edible sub stances,
wherein said ultrasonic waves are focused and directed towards a desired target by means of control of said at least one metasurface.

19. The method of claim 14 wherein said method is used for sonar systems,
wherein, once a target has been identified, said ultrasonic waves are focused and directed towards said target by means of control of said at least one metasurface.
